SEQUENCE LISTING (2 sequences)

SEQ ID NO: 1 - DNA, 19 bp, Artificial Sequence
  Description of Artificial Sequence Primer
ggcacagcacatcaagaga

SEQ ID NO: 2 - DNA, 20 bp, Artificial Sequence
  Description of Artificial Sequence Primer
tgaagatgcggacttacgtg

(19) United States
(12) Patent Application Publication
(10) Pub. No.: US 2005/0044595 A1
(43) Pub. Date: Feb. 24, 2005

(54) In planta transformation by embryo imbibition of agrobacterium

(76) Inventors: Diana Arias, Cary, NC (US); Bryan McKersie, Cary, NC (US); Jean Taylor, Cleveland, TN (US)

Correspondence Address: SUTHERLAND ASBILL & BRENNAN LLP, 999 PEACHTREE STREET, N.E., ATLANTA, GA, 30309, US

(21) Appl. No.: 10/488,088

(22) PCT Filed: Aug. 26, 2002
(86) PCT No.: PCT/US02/27164

Related U.S. Application Data
(60) Provisional application No. 60/314,780, filed on Aug. 24, 2001.

(51) Int. Cl. C12N 15/82; A01H 5/00; A01H 1/00

(52) U.S. Cl. 800/294; 800/278; 800/298

(57) ABSTRACT

The present invention provides a method for the preparation of a plant embryo for Agrobacterium-mediated transformation. The method of preparation uses a novel technique including dehydration of the plant embryo. The invention further contemplates the transformation of the prepared plant embryo. The invention further encompasses the regeneration of a plant or plant cell from the transformed plant embryo.

### BACKGROUND OF THE INVENTION

1. Field of the Invention

This invention generally relates to methods for plant transformation in a genotype-independent manner using plant embryos. These methods use imbibition and desiccation as a novel method for preparing the plant embryo and promoting infection of the tissue by an Agrobacterium. This method of preparing tissues for transformation is useful for preparing both plant zygotic embryos and plant somatic embryos.

2. Background Art

Foreign DNA is usually delivered to a plant nucleus via bombardment-mediated transformation or Agrobacterium-mediated transformation. Bombardment-mediated transformation, or biolistics, is a process by which DNA can be delivered into cells in association with high-velocity microprojectiles (Sanford, 1990 Physiol Plant 79: 206-209; Klein, et al., 1988 Proc Natl Acad Sci USA 85: 8502-8505; Finer and McMullen, 1990 Plant Cell Rep. 8: 586-589). Although several plant species have been transformed by biolistic methods, the frequency of stable transformation can be quite low due to the absence of a mechanism to mediate the integration of the foreign DNA into the plant genome. In addition, bombardment of plant cells with DNA results in the delivery of more than one copy or the partial integration of the gene of interest into the plant cell genome (Hansen and Chilton, 1996 Proc Natl Acad Sci USA 93: 14978-14983), which causes deleterious changes to other traits of the targeted cell.

Unlike biolistic methods, Agrobacterium-mediated transformation does provide a mechanism to mediate the integration of foreign DNA into the plant genome. Agrobacterium is a soil born phytopathogen that integrates a piece of DNA (T-DNA) into the genome of a large number of dicotyledonous and few monocotyledonous plants (Chilton, et al., 1977 Cell 11:263-271; Hoekema, et al., 1985 Nature 303: 179-180; Bevan, 1984 Nucleic Acids Res. 12: 8711-8721; Sheng and Citovsky, 1996 The Plant Cell, Vol. 8. 1699-1710). The T-DNA is flanked by specific sequences, called the right and left borders (Wang, et al., 1987 Science 235: 587-591). The expression of this transferred DNA results in neoplastic growths (tumors) on the host plant. However, because the T-DNA element is defined by its borders, any gene of interest can replace the coding region of the wild type T-DNA. As a consequence, Agrobacterium can be used to produce transgenic plants expressing genes of interest.

Although there are some advantages to using Agrobacterium-mediated transformation instead of biolistic methods, both of those systems depend on the regeneration capacity of the plant cells that have been transformed. Plant regeneration is genotype dependent in most crops and is a labor-intensive task that requires specialized knowledge in the art of tissue culture. Transformation procedures that avoid tissue culture would be extremely valuable, especially for those recalcitrant crops or elite commercial lines that for unknown reasons lack regeneration capacity.

Scientists have attempted to develop plant transformation procedures that do not require tissue culture, but these attempts have been met with limited success. For example, Graves and Goldman (1986 Plant Mol. Biol. 7:43-50) reported that Agrobacterium could infect escutellar and mesocotyl cells of germinating corn seeds, but the resulting transformed plants were chimeras and the transformation efficiency was extremely poor. Additionally, Feldmann and Marks (1987 Mol. Gen. Genet. Vol. 1-2: 1-9) were able to obtain G418 resistant Arabidopsis thaliana plants by co-cultivating germinating seeds with Agrobacterium tumefaciens containing a binary plasmid with a neomycin phosphotransferase (NPT) II gene in its T-DNA region, but the efficiency was again poor. Later, Bechtold et al. (1993, C. R. Acad. Sci. Paris, Sciences de la vie. 316: 1194-1199), reported the transformation of Arabidopsis thaliana by inoculating adult plants with Agrobacterium tumefaciens. Plants infiltrated under a vacuum with a medium containing a concentrated suspension of the bacteria were allowed to grow to maturity in the greenhouse and their seeds harvested and screened for the presence of the foreign DNA. The transformation efficiency reached by Bechtold et al. was two to three orders of magnitude higher than the mean frequency obtained through the seed infection technique described by Feldman and Marks in 1987. However, the Bechtold methodology relies on the size and morphology of the Arabidopsis plant, thereby making the application of the methodology to crops such as soybean or canola difficult or inconvenient to perform.

Since these initial studies, several papers have been published that describe the use of germ line cells as the target cells for transformation, which removes the need for an intermediate tissue culture step. (Chung, et al. 2000 Transgenic Research 9: 471-476, Rohini, et al., 2000 Annals of Botany, 86: 1043-1049; and Trieu., et al., 2000 The Plant Journal, 22(6): 531-541). This system of DNA delivery has been termed In Planta or In Situ transformation. In Planta transformation methods circumvent the difficulty associated with genotype dependent regeneration of elite soybean, maize, canola, cotton, sunflower cultivars common bean, sugar beet, rice, wheat, barley, oil palm, cassaya, and various forest and pine trees from cell cultures and reduce the time required to market commercial transgenic crops.

One prior art In Planta transformation system includes the spraying of an Agrobacterium culture onto plant organs. Chung et al. (2000 Transgenic Research 9: 471-476) demonstrated that spraying the Agrobacterium culture onto immature Arabidopsis flower buds is comparable in transformation efficiency to vacuum-infiltration and floral dip methods. Nevertheless, this prior art method likely cannot be used to successfully transform seedlings in several plant species as shown in Trieu et al. (2000 The Plant Journal, 22(6): 531-541). Trieu et al. reported that the rates of transformation efficiency were 2.8 times lower in the case of Medicago transformation by seedling infiltration when compared to flower infiltration.

Other prior art In Planta transformation methods include the transformation of zygotic embryos. Rohini and Rao (2000 Annals of Botany, 86: 1043-1049) were able to transform safflower by co-cultivating zygotic embryos with the Agrobacterium culture. The embryos were removed from germinating seeds and wounded with a sewing needle prior to co-cultivation. Additionally, Martinell et al. (U.S. Pat. No. 6,384,301) were able to transform soybean embryos by co-cultivating the exposed meristematic tissue of an embryo with Agrobacterium after the tissue had been wounded by ultrasonic waves, a plasma blast discharge, or by puncturing the tissue with a sharp object. Although seemingly successful, these prior art methods involve expensive and/or time-consuming methods of wounding the seed or embryonic tissue.

In addition to transformation of zygotic embryos, the prior art describes the transformation of somatic embryos using naked DNA. A report by Senaratna et al. (1991 Plant Science, 79: 223-228) claims that dry somatic embryos, when imbibed in a solution containing a plasmid carrying the uidA gene, were able to uptake the DNA directly without an Agrobacterium vector. Transient expression of the GUS gene was observed visually in germinating embryos and seedlings. However, this technique has not been readily replicated by other scientists and cannot be used as a reliable method of plant transformation. Moreover, the use of naked DNA by Senaratna et al. is a less efficient method since naked DNA lacks the machinery associated with an Agrobacterium T-DNA in terms of vir genes, mobilization and integration proteins.

There is a need, therefore, to identify a rapid and genotype-independent method of transformation that is applicable to any plant species that can provide a zygotic or somatic embryo and is capable of germination after imbibition in Agrobacterium suspension cultures.

### SUMMARY OF THE INVENTION

It is an object of the present invention to overcome, or at least alleviate, one or more of the difficulties or deficiencies associated with the prior art. In that regard, the present invention provides a rapid, genotype-independent, and cell culture-free method for delivering transforming agents to germ line tissues, which overcomes deficiencies of the prior art methods. In particular, the invention provides for a method of preparing a cell for transformation, and provides for a method of transforming the cell, and regenerating the transformed cell into a plant or a plant part.

One preferred embodiment comprises a method of preparing a plant zygotic embryo for transformation comprising: (a) imbibing the plant zygotic embryo; and (b) dehydrating the plant zygotic embryo. In one embodiment, the plant zygotic embryo of step (a) is a seed. In a further or optional embodiment, the plant zygotic embryo of step (b) is a seed. In some embodiments, the transformed plant zygotic embryo is regenerated into a transgenic plant or a transgenic plant tissue. In another preferred embodiment, the invention encompasses a method of preparing a somatic embryo for transformation comprising dehydrating the somatic embryo.

The methods of the current invention are useful in the transformation of plant embryos. Examples of plant embryos are zygotic embryos, and somatic embryos.

The present invention further encompasses the transformation of the dehydrated plant zygotic embryo. The present invention provides a method of transforming a plant embryo, including the steps of (a) imbibing the plant zygotic embryo in an aqueous solution; (b) dehydrating the plant zygotic embryo; and (c) imbibing the plant zygotic embryo in an Agrobacterium solution wherein the Agrobacterium comprises a transgene. In another embodiment, the invention provides a method of transforming a somatic embryo, comprising the steps of (a) dehydrating the somatic embryo; and (b) imbibing the plant somatic embryo in an Agrobacterium solution wherein the Agrobacterium comprises a transgene. In preferred embodiments, transformation comprises imbibing the dehydrated plant embryo with an Agrobacterium solution wherein the Agrobacterium comprises a transgene. In further preferred embodiments, the transgene encodes a protein that alters the phenotype of the transformed plant. The transgene can optionally comprise a herbicide resistance gene.

The above and below methods can be used to stably integrate a gene into the germ-line of a zygotic plant embryo without the necessity of going through the tissue culture process, which is a major advantage over the prior art. The plant embryo can be from a monocotyledonous or dicotyledonous plant. In a preferred embodiment, the plant embryo is derived from a soybean plant or a canola plant. The invention also provides for the transgenic plants generated using these methods. In further embodiments, progeny of the transgenic plant are selected that also contain the transgene.

### BRIEF DESCRIPTION OF THE DRAWINGS

FIG. 1 is a table that shows the germination rates of soybean embryo axes after different periods of imbibition and dehydration. Moisture content (%) is calculated as grams water/grams fresh weight multiplied by 100%.

FIG. 2 is a table that shows examples of binary vectors used in this invention.

FIG. 3 is a table that shows a visual analysis of Gus expression in soybean tissues after In Planta transformation by embryo imbibition of Agrobacterium.

FIG. 4 is a table that shows that 19 out of 29 T3 soybean plants transformed via the embryo imbibition of Agrobacterium amplified the 500 base pair band that corresponds to a fragment of the uidA gene

FIG. 5 is a table that shows a histological analysis of uidA gene expression in canola seedlings after Agrobacterium imbibition.

### DETAILED DESCRIPTION OF THE INVENTION

The present invention provides a method for preparing plant zygotic and somatic embryos for transformation, and provides a method for transforming plant zygotic and somatic embryos. The methods of the invention provide several advantages over previously available methods. For example, the methods of the invention providing for zygotic embryo transformation do not require tissue culture procedures which is a major advantage over the prior art. This method allows for a more rapid and more efficient transformation process than previously used techniques. In addition, the process of imbibition and dehydration provides a method for plant transformation that is genotype-independent, and cell-culture independent, thus avoiding the problem of somaclonal variation. Some methods of the invention include imbibition of the embryo, and a subsequent dehydration of the embryo prior to transformation of the embryo. It is believed that the dehydration process results in an increase in the secretion of phenolic compounds, which facilitates the Agrobacterium infection. It is also believed that these phenolic compounds stimulate the Agrobacterium vir gene expression required for initiation of T-DNA transport into the plant cell nucleus (Citovsky and Zambryski, 1993 Ann. Rev. Microbiol. 47, 167-197).

The present invention particularly provides a method of preparing a plant zygotic embryo for transformation comprising: (a) imbibing the plant zygotic embryo in an aqueous solution; and (b) dehydrating the plant zygotic embryo. In another embodiment, the invention provides a method of preparing a somatic embryo for transformation comprising dehydrating the somatic embryo. The present invention additionally provides a method of transforming the plant zygotic embryo, including the steps of (a) imbibing the plant zygotic embryo in an aqueous solution; (b) dehydrating the plant zygotic embryo; and (c) imbibing the plant zygotic embryo in an Agrobacterium solution wherein the Agrobacterium comprises a transgene. In an alternative embodiment, the invention provides a method of transforming a somatic embryo, comprising the steps of (a) dehydrating the somatic embryo; and (b) imbibing the plant somatic embryo in an Agrobacterium solution wherein the Agrobacterium comprises a transgene. In one embodiment, the transformed plant embryo is regenerated into a transgenic plant or a transgenic plant tissue.

Unless otherwise noted, the terms used herein are to be understood according to conventional usage by those of ordinary skill in the relevant art. In addition to the definitions of terms provided below, definitions of common terms in molecular biology may also be found in Rieger et al., 1991 Glossary of genetics: classical and molecular, 5th ed, Berlin: Springer-Verlag; and in Current Protocols in Molecular Biology, F. M. Ausubel et al., eds., Current Protocols, a joint venture between Greene Publishing Associates, Inc. and John Wiley & Sons, Inc., (1998 Supplement). It is to be understood that as used in the specification and in the claims, “a” or “an” can mean one or more, depending upon the context in which it is used. Thus, for example, reference to “a cell” can mean that at least one cell can be utilized.

As used herein, the term “plant embryo” includes both a zygotic embryo, and a somatic embryo. As used herein, the term “zygotic embryo” refers to the product of the fusion of male and female gametes. The term “zygotic embryo” is to be understood to encompass an embryonic axis, an embryonic axis with the cotyledons wholly or partially removed, part of a seed, or an entire seed.

As also used herein, the term “somatic embryo” refers to an embryo derived or induced from the vegetative part of a plant, which is the part of the plant not originally destined to become a gamete. Plant regeneration through somatic embryogenesis is the currently preferred process for some plant species. Somatic embryos can be induced from different types of plant tissues, including, but not limited to, an immature zygotic embryo, a leaf, a node, an internode, a shoot, an axillary bud, a shoot meristem, a root meristem, a cotyledon, a petiole, a microspore, a flower petal and a hypocotyl. The highest somatic embyogenic capacity is typically found in immature tissues, such as, but not limited to, the immature cotyledon. The medium used to induce the formation of a somatic embryo typically comprises inorganic salts, a carbon source such as sucrose, inositol, thiamine, and hormones. The composition of such plant tissue culture media may be modified to optimize the growth of the particular plant cells employed. The cell type and specific culture conditions including hormones that can be used to derive somatic embryos can vary with the plant specie. For example, a preferred embodiment used to derive somatic embryos from a soybean plant encompasses the use of immature cotyledons as the source of the somatic tissue, and the use of the hormone 2,4-D in the tissue culture conditions. Once the system of regeneration has been established, a somatic embryo produced therefrom can be considered an analog of a zygotic embryo. The present invention is therefore applicable to those species that routinely undergo somatic embryogenesis, such as carrots, alfalfa, sugar beet, rice, cyclamen, tomato, cucumber, soybean, corn, wheat, barley, cassaya, ginseng, banana, pea, and pepper, among others.

As stated above, the present invention provides a method of preparing a plant zygotic embryo for transformation comprising: (a) imbibing the plant zygotic embryo in an aqueous solution; and (b) dehydrating the plant zygotic embryo. In some embodiments of the present invention, the plant zygotic embryo of both step (a) and (b) is a seed. In these embodiments, a plant seed is prepared for transformation by imbibing the seed in an aqueous solution and then dehydrating the seed to a particular moisture content. The seed is then transformed by imbibing the seed in an Agrobacterium solution wherein the Agrobacterium comprises a transgene. When the seed is dehydrated in an intact form, in one embodiment, the intact dehydrated seed is imbibed with the Agrobacterium solution. Alternatively, portions of the intact seed are imbibed with the Agrobacterium solution. In other embodiments of the present invention, the plant zygotic embryo of step (a) is a seed, but the plant zygotic embryo of step (b) is not a seed. In these embodiments, a plant seed is prepared for transformation by imbibing the seed in an aqueous solution, the seed coat is removed, one or both cotyledons are excised to expose the embryonic axis, and the exposed plant tissue comprising the embryonic axis (or plant zygotic embryo) is dehydrated. In another embodiment, the invention provides a method of preparing a plant somatic embryo for transformation, comprising dehydrating the somatic embryo.

The plant embryo used in the present invention can be from any plant, including but not limited to, maize, wheat, rye, oat, triticale, rice, barley, soybean, peanut, cotton, rapeseed and canola, manihot, pepper, sunflower and tagetes, solanaceous plants like potato, tobacco, eggplant, and tomato, Vicia species, pea, alfalfa, bushy plants (coffee, cacao, tea), Salix species, trees (oil palm, coconut), perennial grasses and forage crops. In preferred embodiments, the plant is selected from the group consisting of a legume (i.e., soybean, alfalfa, common bean etc.), maize, wheat, rice, barley, canola, sugar beet, tagetes, sunflower, or cotton plant. In a further preferred embodiment, the plant embryo is from canola, or soybean.

As used herein, the term “imbibition” or “imbibing” refers to the absorption of a liquid by the plant embryo. The plant embryo is placed in a liquid, or in positioned such that it is in contact with a liquid, such that the plant embryo absorbs the liquid. In one embodiment, the plant zygotic embryo is imbibed in an aqueous solution. In a preferred embodiment, the aqueous solution comprises water. In a further preferred embodiment, the aqueous solution consists essentially of water. In other embodiments, the aqueous solution further comprises additives, such as hormones (cytokinins, auxins, gibberellins) minerals (macro and micronutrients), vitamins, and surfactants (such as Tween™). The plant zygotic embryo is imbibed in an aqueous solution for approximately 6-39 hours, more preferably approximately 15-24 hours, and most preferably approximately 17-19 hours. In one embodiment, the plant zygotic embryo is imbibed in an aqueous solution for approximately 18 hours. In other embodiments the plant zygotic embryo is imbibed in an aqueous solution for approximately 15, 16, 17, 18, 19, 20, 21, 22, 23, 24, or 25 hours. In another embodiment the plant zygotic embryo is imbibed for no more than 48 hours. The plant zygotic embryo is imbibed in an aqueous solution at approximately 15-30° C. In a preferred embodiment, the plant zygotic embryo is imbibed in the aqueous solution at room temperature.

As used herein, the terms “dehydrate” or “dessicate” are used interchangeably, and refer to a reduction in the water or moisture content of the plant embryo. The plant embryo may be dehydrated by placing the embryo under a laminar flow hood for various periods of time. Moisture content is expressed as a percentage, and is calculated as grams of water in the dehydrated plant embryo divided by the weight before dessication, also called its fresh weight, multiplied by 100%. The water content is determined as described by Senaratna and McKersie, 1983 Plant Physiol. 72: 620-624. In preferred embodiments, the plant embryo is dehydrated to a moisture content of approximately 0-60%, more preferably approximately 10-25%, and most preferably approximately 15-25%. In one embodiment, the moisture content is less than approximately 20%. In other embodiments, plant tissue is dehydrated to a moisture content of approximately 10%, 11%, 12%, 13%, 14%, 15%, 16%, 17%, 18%, 19%, 20%, 21%, 22%, 23%, 24%, or 25%.

The present invention further encompasses the transformation of the plant embryo. In a preferred embodiment, transformation comprises imbibing the dehydrated plant embryo with an Agrobacterium solution wherein the Agrobacterium comprises a transgene. The invention contemplates that the imbibition time with the Agrobacterium solution can vary. It is generally preferred that the plant embryo is imbibed in an Agrobacterium solution for approximately 0.5 to 3 hours, and more preferably for approximately 1-2 hours. In one embodiment the plant embryo is imbibed in an Agrobacterium solution for at least 30 minutes, preferably for approximately 1 hour, and more preferably for approximately 90 minutes. In a preferred embodiment, the plant embryo is imbibed in Agrobacterium solution for approximately two hours.

In a preferred embodiment of the present invention, a plant zygotic embryo is imbibed with an aqueous solution for 15-24 hours, and preferably for approximately 18 hours. The plant zygotic embryo is then dehydrated at room temperature overnight or until the plant zygotic embryo is dehydrated to a moisture content of 10-25%, or preferably to a moisture content of less than approximately 20%. The dehydrated plant zygotic embryo is then transformed with an Agrobacterium solution for approximately 2 hours at room temperature, wherein the Agrobacterium comprises a transgene. In one embodiment a seed containing the plant zygotic embryo is used in the first imbibition step. The plant zygotic embryo may or may not then be dissociated from the rest of the seed, or portions of the seed, before dehydration. For example, the plant zygotic embryo may be dissociated from the cotyledons or portions of the cotyledons. The imbibition and dehydration times are varied in order to optimize the conditions for germination and for genetic transformation of a plant zygotic embryo derived from a specific plant species.

In another preferred embodiment, a plant somatic embryo is dehydrated at room temperature overnight or until the plant somatic embryo is dehydrated to a moisture content of 10-25%, or preferably to a moisture content of less than approximately 20%. The dehydrated plant somatic embryo is then transformed with an Agrobacterium solution for approximately 2 hours at room temperature, wherein the Agrobacterium comprises a transgene. The imbibition and dehydration times are varied in order to optimize the conditions for germination and for genetic transformation of a plant somatic embryo derived from a specific plant species.

The invention contemplates the use of an Agrobacterium solution to transform the plant embryo wherein the Agrobacterium comprises a transgene. In one embodiment, the Agrobacterium solution is a culture medium, wherein the medium comprises MSB5 (Murashige and Skoog salts and Gamborgs B5 vitamins) and acetosyringone. In a preferred embodiment, acetosyringone is present at a concentration of approximately 100 μM. In another embodiment, the Agrobacterium solution does not comprise acetosyringone. In another or further embodiment, the Agrobacterium solution comprises a phenolic compound, including, but not limited to, α-hydroxyacetosyringone, acetovanillone, syringaldehyde, syringic acid, and sinapinic acid. In one embodiment, the plant embryo is not germinated prior to incubation with the Agrobacterium solution. Various strains of Agrobacterium having different chromosomal backgrounds and Ti-plasmid content can be used for the Agrobacterium solution. However, it is preferred that the Agrobacterium strain contains a disarmed Ti-plasmid. Agrobacterium strains that can be used include, but are not limited to, LBA4404, GV2260, GV3600, EHA101, EHA105, AGL-1, LBA9402, GV3101, COR341, COR356, UIA143, pCH32, BIBAC2, C58C1, pMP90 and AGT121. In a preferred embodiment the Agrobacterium strain is selected from the group consisting of C58C1, pMP90, and LBA4404.

As used herein, “transformed” refers to a cell, tissue, or organism into which a transgene, such as a recombinant vector, has been introduced. Such a cell, tissue, or organism is considered “transformed” or “transgenic,” as is progeny thereof in which the foreign nucleic acid or transgene is present. The method of the invention can be used to “prepare” a plant embryo in order to facilitate transformation by any transformation method known to those of skill in the art. Methods that can be used to transform the prepared plant embryo include Agrobacterium mediated transformation, microprojectile bombardment, microinjection, macroinjection, polyethylene glycol (PEG) treatment of protoplasts, and liposome-mediated DNA uptake. These methods are described in, for example, B. Jenes et al., and W. W. Ritchie et al. In Transgenic Plants, Vol. 1, Engineering and Utilization, ed. S. D. Kung, R. Wu, Academic Press, Inc., Harcourt Brace Jovanovich 1993; and L. Mannonen et al., Critical Reviews in Biotechnology, 14:287-310, 1994). “Foreign” nucleic acids are nucleic acids that would not normally be present in the host cell, referring, in particular, to nucleic acids that have been modified by recombinant DNA techniques. The term “foreign” nucleic acids also includes host genes that are placed under the control of a new promoter or terminator sequence, for example, by conventional techniques.

In a preferred embodiment, the Agrobacterium solution comprises a transgene. In one embodiment, the transgene encodes a protein that alters the phenotype of the transformed plant. As used herein, the term “alters” refers to the expression of a gene that adds, deletes, or modifies a phenotypic trait. The transgene can comprise any gene, but preferably the transgene comprises a gene for a selectable marker. In one preferred embodiment, the selectable marker is a gene encoding for herbicide resistance. Examples of herbicide resistance genes include, but are not limited to the gene encoding the enzyme 5-enolpyruvylshikimate-3-phosphate synthase (EPSPS), a gene encoding the enzyme phosphinothricin acetyl transferase (PAT), and a gene encoding a mutant acetohydroxyacid synthase (AHAS) enzyme. The Agrobacterium vector can contain a selectable marker, a promoter, a polyadenylation sequence, and a signal peptide. Construction of the vector can be performed by ligation of the gene of interest in a sense or antisense orientation into the T-DNA. Located 5-prime to the cDNA, a plant promoter can be used to activate transcription of the cDNA. A polyadenylation sequence may be located 3-prime to the cDNA.

Tissue-specific expression of the transgene can be achieved by using a tissue specific promoter. For example, seed-specific expression can be achieved by cloning the napin or LeB4 or USP promoter 5-prime to the cDNA. Also, any other seed specific promoter element can be used. For constitutive expression within the whole plant, the CaMV 35S promoter can be used. One skilled in the art will recognize that the promoter used should be operatively linked to the nucleic acid such that the promoter causes transcription of the nucleic acid which results in the synthesis of a mRNA which encodes a polypeptide. Alternatively, the RNA can be an antisense RNA for use in affecting subsequent expression of the same or another gene or genes.

The invention further contemplates that after the plant embryo is imbibed in the Agrobacterium solution, the plant embryo is transferred to an incubation medium. The incubation medium comprises plant culture medium. As used herein, “plant culture medium” refers to any medium used in the art for supporting viability and growth of a plant cell or tissue, or for growth of whole plant specimens. Such media commonly include defined components including, but not limited to: macronutrient compounds providing nutritional sources of nitrogen, phosphorus, potassium, sulfur, calcium, magnesium, and iron; micronutrients, such as boron, molybdenum, manganese, cobalt, zinc, copper, chlorine, and iodine; carbohydrates; vitamins; phytohormones such as auxins, cytokinins, and giberellins; selection agents (for transformed cells or tissues, e.g., antibiotics or herbicides); and gelling agents (e.g., agar, Bactoagar, agarose, Phytagel, Phytagar, Gelrite, etc.); and may include undefined components, including, but not limited to: coconut milk, casein hydrolysate, yeast extract, and activated charcoal. In a preferred embodiment, the incubation medium comprises an essentially Agrobacterium free incubation medium. As used herein, “essentially Agrobacterium free” refers to a medium that does not comprise an Agrobacterium prior to the time when the plant embryo is transferred to the medium, or to a medium that comprises a de minimus amount of Agrobacteria prior to the time the plant embryo is transferred to the medium. For example, an essentially Agrobacterium free incubation medium can contain less than 10%, 9%, 8%, 7%, 6%, 5%, 4%, 3% 2% or 1% Agrobacteria. In one embodiment, the incubation medium is a solid medium, or alternatively, it is a semi-solid medium. In a preferred embodiment, the incubation medium is a MS medium.

The invention contemplates that after the plant embryo is transferred to the incubation medium, and before it is incubated in a further growth medium, the plant embryo can be treated with an Agrobacterium inhibiting agent. Treatment can consist of washing the plant embryo to remove the Agrobacterium or applying a chemical agent the inhibits an Agrobacterium. By “inhibiting” or “inhibits” it is meant the agent can remove an Agrobacterium from the exterior of the plant embryo, the agent can inhibit the ability of an Agrobacterium to infect the plant embryo, or alternatively, the agent can kill at least a percentage of the Agrobacteria surrounding the plant embryo. In one example, the agent inhibits at least 40%, 50%, 60%, 70%, 80%, 90%, 95%, 96%, 97%, 98%, 99% or 100% of the Agrobacteria. In one embodiment, the Agrobacterium inhibiting agent is selected from the group consisting of timentin, carbenicillin, augmentin, varnecillin and cefotaxime. In a preferred embodiment the Agrobacterium inhibiting agent is timentin. Preferably, the concentration of timentin is approximately 1-1000 mg/l, more preferably approximately 50-750 mg/L, or most preferably approximately 400-600 mg/L. In one embodiment, timentin is used at a concentration of approximately 500 mg/L.

In a preferred embodiment of the present invention, the plant zygotic embryo is imbibed with an aqueous solution for 15-24 hours, and preferably for approximately 18 hours. The plant zygotic embryo is then dehydrated at room temperature overnight or until the plant zygotic embryo is dehydrated to a moisture content of 10-25% or preferably to a moisture content of less than approximately 20%. The dehydrated plant zygotic embryo is then transformed with an Agrobacterium solution wherein the Agrobacterium comprises a transgene for approximately 2 hours at room temperature. After the imbibition with the Agrobacterium solution, the plant zygotic embryo is transferred to an incubation medium for 1-3 days, and preferably approximately 2 days, wherein the incubation medium is preferably an essentially Agrobacterium free incubation medium. After incubating on the incubation medium for a sufficient amount of time to facilitate infection by the Agrobacterium, the plant zygotic embryo is treated with an effective amount of an Agrobacterium inhibiting agent. In one embodiment, the Agrobacterium inhibiting agent is timentin, wherein the effective amount of timentin is approximately 500-mg/L.

In another preferred embodiment, a plant somatic embryo is dehydrated at room temperature overnight or until the plant somatic embryo is dehydrated to a moisture content of 10-25%, or preferably to a moisture content of less than approximately 20%. The dehydrated plant somatic embryo is then transformed with an Agrobacterium solution for approximately 2 hours at room temperature, wherein the Agrobacterium comprises a transgene. After the imbibition with the Agrobacterium solution, the plant somatic embryo is transferred to an incubation medium for 1-3 days, and preferably approximately 2 days, wherein the incubation medium is preferably an essentially Agrobacterium free incubation medium. After incubating on the incubation medium for a sufficient amount of time to facilitate infection by the Agrobacterium, the plant somatic embryo is treated with an effective amount of an Agrobacterium inhibiting agent. In one embodiment, the Agrobacterium inhibiting agent is timentin, wherein the effective amount of timentin is approximately 500 mg/L.

The invention further contemplates the regeneration of a transgenic plant, or plant tissue, from the transformed plant embryo produced by the methods described above. As used herein, the term “plant” encompasses transgenic plants, and progeny of such plants. As also used herein, the term “plant tissue” refers to a part of a plant, including a plant organ, or any group of plant cells organized into a structural or functional unit. The term “tissue” is to be understood to be composed of several cells, for example, more than one cell. The term “plant organ” refers to a distinct and visibly differentiated part of a plant, such as a root, a stem, a leaf or an embryo. The term “regeneration” as used herein refers to the production of at least one newly developed or regenerated plant tissue, e.g., root, shoot, callus, etc., from a plant embryo. Accordingly, the invention further provides for a transgenic plant or plant tissue produced using any of the above or below methods.

Transgenic plant embryos, transgenic plant tissues or transgenic plants may be selected for using a selection agent. As used herein, the term “selection agent” refers to a compound that when applied to plant embryos, or plant tissues or plants not containing a particular transgene, results in the death or injury of those plant embryos, plant tissues or plants. Plant embryos, plant tissues and plants containing the transgene, or selectable marker gene, survive the application of the selection agent, and therefore, are “selected”. The selection agent can be a metabolic inhibitor, an antibiotic, herbicide or the like. In one embodiment the selection agent is a herbicide. In a preferred embodiment, the transformed plant embryo is placed on an incubation medium for approximately 2 days, it is then subsequently treated with an Agrobacterium inhibiting agent, and is placed on a further growth medium comprising a selection agent.

The process of producing a new plant from a zygotic embryo can encompass shoot development or germination. The process of regeneration may be preferably applied to somatic embryogenesis. Regeneration of a transgenic plant can begin by placing the transformed plant embryo in a plant growth medium. In one embodiment, the medium is a MS or N6 medium that can be modified by including further substances such as carbon sources, salts, and hormones. A typical hormone for such purposes is dicamba or 2,4-D. However, other hormones may be employed, including NAA, NAA and 2,4-D, or picloram. In one embodiment, the medium that supports the regeneration of plants is MS medium used with vermiculite supplemented with an Agrobacterium inhibiting agent. In a preferred embodiment, the Agrobacterium inhibiting agent is timentin. Cells are typically maintained on this media with or without hormones until sufficient tissue is available to begin plant regeneration efforts, or if following repeated rounds of manual selection, until the morphology of the tissue is suitable for regeneration. The tissue is then transferred to a medium conducive to maturation of the tissue. Once shoot induction has begun, the cultures are transferred to a medium that does not contain hormones.

The cultures are then allowed to mature into plants. Developing plantlets are transferred to plant growth mix, and hardened. In a preferred embodiment, the plant growth mix is metromix media. Plants are preferably matured either in a growth chamber or greenhouse. Regenerating plants are preferably grown at approximately 19 to 28° C., and more preferably at approximately 25° C. After the regenerating plants have reached the stage of shoot and root development, they may be transferred to a greenhouse for further growth and testing.

Throughout this application, various publications are referenced. The disclosures of all of these publications and those references cited within those publications in their entireties are hereby incorporated by reference into this application in order to more fully describe the state of the art to which this invention pertains. The following examples are not intended to limit the scope of the claims to the invention, but are rather intended to be exemplary of certain embodiments.

### EXAMPLES

### Example 1

Soybean

Soybean seeds were surface sterilized with 70% ethanol for 4 minutes with continuous shaking, followed continuous shaking in 20% (v/v) Clorox™ supplemented with 0.05% (v/v) Tween 20™ for 20 minutes. Unless otherwise indicated, these examples were performed at room temperature. The seeds were then rinsed 4 times with distilled water and placed on moistened sterile filter paper in a Petri dish at room temperature for 6 to 39 hours. The seed coats were peeled off, and one or both cotyledons were detached from the embryo axis. The embryo axis was examined to make sure that the meristematic region was not damaged. The excised explants were collected in a half-open sterile Petri dish and air-dried. During this period, the embryo loses approximately 40 to 90% of its water content.

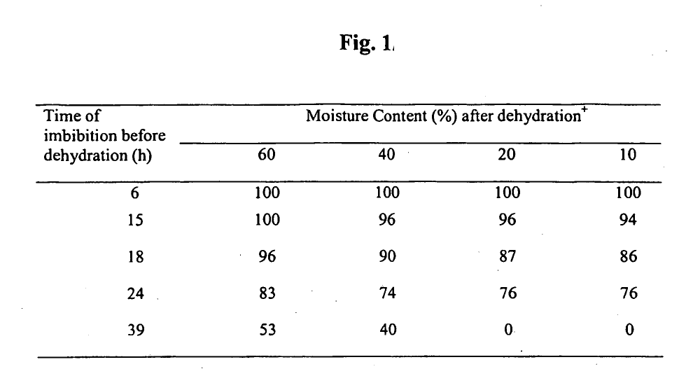

In one experiment, the soybean seeds were imbibed in water for 6 to 39 hours before the seed coats were removed and the embryo axes (with no cotyledons or with one cotyledon) were excised, dehydrated to various water contents and then germinated in sterile filter paper pre-wetted with MSB5 media. FIG. 1 shows the average germination rates of two replications of this experiment. The germination of the embryo axes was not affected by any dehydration treatment after 6 hours of imbibition, and was affected only slightly after 15 hours of imbibition. On the other hand, embryo axes that had lost 60 to 90% of their water content showed a decreased germination rate when the embryo axes were imbibed in water for 24 or more hours. Since the purpose of the experiment was to facilitate Agrobacterium infection, the time period selected for the imbibition of embryo axes was 18 hours imbibition followed by overnight dehydration. Consequently, the routine procedure followed was to store the embryo axes at room temperature (RT) after being air-dried to a moisture content less than 20% (fresh weight) in a sealed Petri dish until further use.

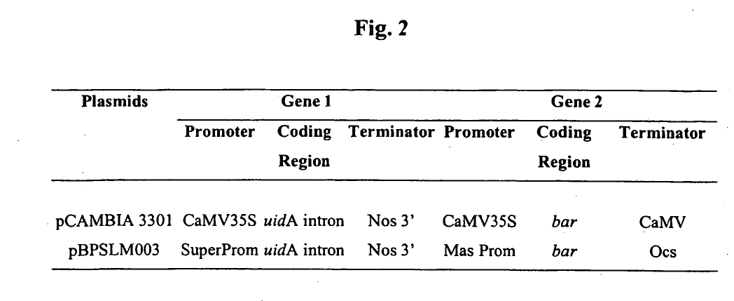

Generally, a T-DNA fragment of a binary vector comprises two transgenes. In this specific example, one transgene was operatively linked to a constitutive promoter for expression of the selectable marker, i.e. bar. The selectable marker confers resistance to glufosinate-type herbicides, such as Liberty™, phosphinothricin (PPT) or bialaphos. The other transgene may include a reporter gene such as the uidA gene (See FIG. 2). A binary vector harboring each of the T-DNA's described previously is transformed into an Agrobacterium tumefaciens strain (e.g. C58C1, pMP90, or LBA4404) following general molecular biology techniques (Sambrook et al. 1989 Molecular Cloning: A Laboratory Manual, Cold Spring Harbor Laboratory Press, Cold Spring Harbor, N.Y.).

Agrobacterium tumefaciens culture was prepared from a single colony in LB solid medium plus appropriate antibiotics (e.g. 100 mg/l streptomycin, 50 mg/l kanamycin), followed by growth of the single colony in liquid LB medium to an optical density (OD) at 600 nm of 0.8. Once the bacteria culture reached the specific OD, the culture was centrifuged at 5000 rpm for 5 minutes and resuspended in MSB5 (Murashige and Skoog salts and Gamborgs B5 vitamins) and the medium was supplemented with 100 μM acetosyringone. Bacteria cultures were incubated in this pre-induction medium for 2 hours at RT before use. The axis of soybean zygotic seed embryos having approximately 15% moisture content were imbibed for 2 hours at RT with the pre-induced Agrobacterium suspension culture. The embryo axes were removed from the imbibition culture and were transferred to Petri dishes containing solid MS (Murashige and Skoog, 1962 Physiol. Plant, 15: 473-479) medium supplemented with 2% sucrose and incubated for 2 days in the dark at RT. After this incubation period, the embryo axes were washed with MS medium supplemented with 500 mg/L timentin to kill or at least inhibit the Agrobacteria. After washing, the embryo axes were incubated in sterile vermiculite pre-wetted with MS medium supplemented with 500 mg/L timentin and incubated for 4 weeks at 25° C., under 150 μmolm⁻² sec⁻¹ with a 12 hour photoperiod. Once the seedlings produced roots, they were transferred to metromix media in a growth cabinet where they were incubated at 25° C., under 380 μmol m⁻² sec⁻¹ light intensity and with a 12 hour photoperiod for approximately 90 days. The plants were kept under a plastic cover for 1 week to favor the acclimatization process. Seeds were collected and planted again to screen for herbicide resistant progeny.

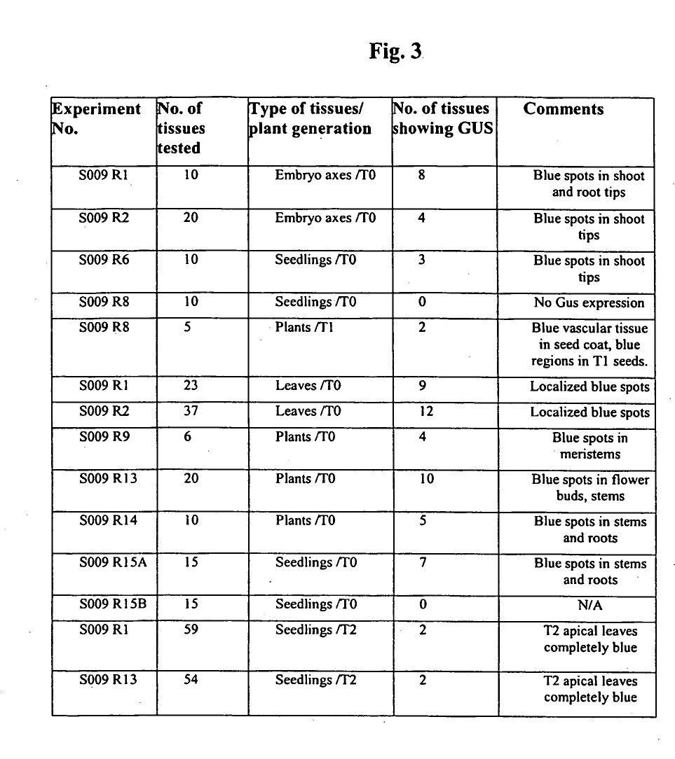

Expression of the uidA gene was evaluated after Agrobacterium imbibition at different stages of the seedling or plant development to determine the integration pattern of the T-DNA. A sample of the explants was visually screened for GUS activity by staining them with the chromogenic substrate X-Gluc (Jefferson, 1987 Plant Molecular Biology Reporter, 5: 387-405). The explants were incubated overnight in a solution containing 50 μg X-Gluc (Research Organics) in 10 mM EDTA, pH 8.0, 100 mM sodium phosphate, pH 7.0, 5 mM potassium ferrocyanide, 5 mM potassium ferrycyanide and 1 μl Triton X-100 for 16-24 hours at 37° C. FIG. 3 describes the different organs or plant regions in which GUS expression was visually detected.

The pCAMBIA3301 and pBPSLM003 vectors have a uidA gene with an intron that prevents its expression in the Agrobacterium. Therefore, positive GUS activity in soybean axes incubated with these T-DNAs indicates that the plant cell was expressing the T-DNA, and that the expression of GUS was not from contaminating Agrobacterium cells.

Samples of the transgenic plants (T1 and following generations) were analyzed by PCR to confirm the presence of T-DNA. Genomic DNA was extracted from a leaf punch using the DNEASY Plant Mini QIAGEN™ kit. Plant tissue was disrupted via the BIO101 Fast Prep machine in 400 μl buffer AP1 supplemented with 4 μl RNase A (100 mg/ml). The homogenate was incubated at 65° C. for 20 minutes. All the other steps were followed according to the manufacturer's instructions. DNA was eluted twice with 50 μl buffer AE. PCR was used to detect the presence of the uidA gene that was introduced into soybean via the Agrobacterium T-DNA. PCR reactions were performed in a volume of 50 μl of 1×PCR buffer+Mg²⁺ (Roche Molecular Biochemicals™ cat # 1815105), 50 to 100 ng of plant DNA, 0.15 μM of each primer, 100 μM dNTP's and 2.5 unit Taq polymerase (Roche Molecular Biochemicals™). The primers (GUSJT1: 5′GGCACAGCACATCAAGAGA3′ (SEQ ID NO:1) and GUSJT2: 5′TGAAGATGCGGACTTACGTG3′ (SEQ ID NO:2)) were synthesized by IDT and amplify a 500 base pair fragment of the uida gene. Transgenic canola transformed with a uidA gene was used as a positive control for the reactions.

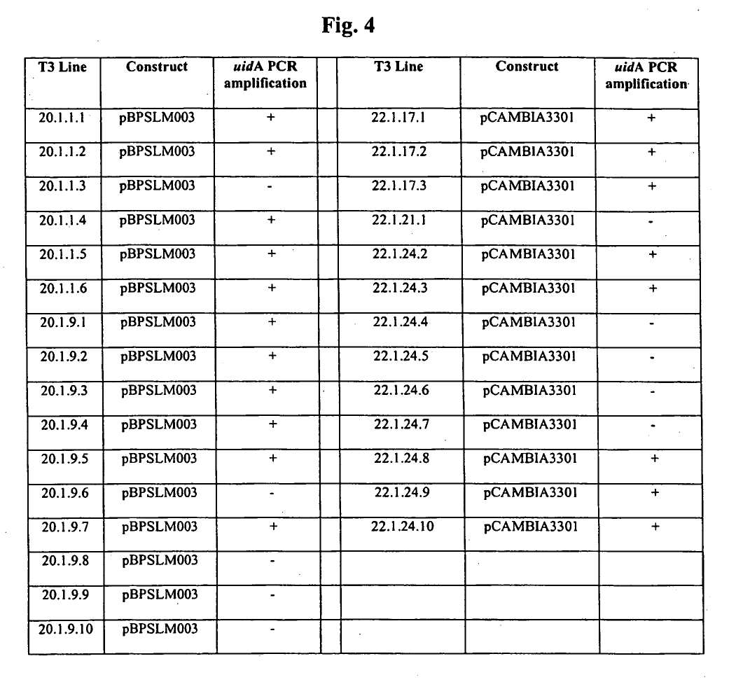

DNA was amplified in a Biometra T gradient thermocycler™. Template DNA was denatured at 94° C. for 4 minutes, followed by 30 cycles of a denaturing step at 94° C. for 50 seconds, an annealing step at 55° C. for 45 seconds and an extension step at 72° C. for 1 minute. The DNA amplification was finished by one cycle of 10 minutes at 72° C. Aliquots were taken directly from the reaction samples and were run on a 1% (w/v) agarose gel containing 0.5 μg/ml ethidium bromide for visualization under UV light. Nineteen out of twenty-nine T3 transgenic soybean plants transformed by embryo imbibition of Agrobacterium amplified the 500 base pair band that corresponds to a fragment of the uidA gene (FIG. 4).

These results were confirmed by Southern hybridization in which 5-10 μg of soybean DNA was electrophoresed on a 1% agarose gel and transferred to a positively charged nylon membrane (Roche Diagnostics). The PCR DIG Probe Synthesis Kit (Roche Diagnostics) was used to prepare a digoxigenin-labelled probe by PCR, and was used as recommended by the manufacturer.

Inheritance and expression of the T-DNA was confirmed in the T1 plants and following generations by testing leaves from each of the plants for activity of the selectable marker. Only a small portion of the T1 plants contained the T-DNA but these were easily selected by performing a herbicide tolerance test. The adaxial surface of a unifoliate leaf of a plant two to three weeks old was painted with a 100 mg/L solution of glufosinate. Herbicide tolerance was monitored 5 days post application. The transgene was transmitted in a Mendelian manner to the T2 and subsequent generations.

### Example 2

Canola

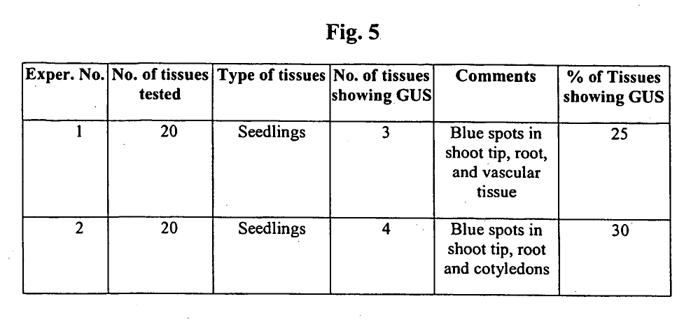

The method of plant transformation described in Example 1 is also applicable to Brassica and other crops. To illustrate this principle, seeds of canola were surface sterilized with 70% ethanol for 4 minutes at RT with continuous shaking, followed by continuous shaking in 20% (v/v) Clorox™ supplemented with 0.05% (v/v) Tween 20™ for 20 minutes at RT. The seeds were then rinsed 4 times with distilled water and placed on moistened sterile filter paper in a Petri dish at room temperature for 18 hours. Then the seed coats were removed and the seeds were air dried overnight in a half-open sterile Petri dish. During this period the seeds lost approximately 85% of their water content. The seeds were then stored at RT in a sealed Petri dish until further use. DNA constructs, embryo axis imbibition and in situ uidA gene expression were as described in Example 1. The histological analysis of the uidA gene expression in canola is shown in FIG. 5.

Samples of the primary transgenic plants (T0) were analyzed by PCR to confirm the presence of T-DNA. These results were confirmed by Southern hybridization in which DNA was electrophoresed on a 1% agarose gel and transferred to a positively charged nylon membrane (Roche Diagnostics). The PCR DIG Probe Synthesis Kit (Roche Diagnostics) was used to prepare a digoxigenin-labelled probe by PCR, and was used as recommended by the manufacturer.

Inheritance and expression of the T-DNA is confirmed in the T1 generation by testing leaves from each of the plants for activity of the uidA reporter gene. Only a small proportion of the T1 plants contain the T-DNA but these are easily selected by spraying the seedlings with a selection agent, such as Basta. The transgene is stably transmitted to the T2 and subsequent generations in a Mendelian manner.

### Example 3

Arabidopsis

This method of transformation is also applicable to whole intact seeds as shown in this example using Arabidopsis. Seeds of Arabidopsis thaliana are surface sterilized as explained in Example 1. The seeds are then rinsed 4 times with distilled water and placed on moistened sterile filter paper in a Petri dish at room temperature for up to 40 hours. The imbibed seeds are collected in a half-open sterile Petri dish and air dried. During this period the embryo may lose up to 90% of its water content.

In one experiment, the seeds are imbibed in water for 6, 12, 18, 24 or 36 hours, and dehydrated to various water contents. Some seeds were immediately placed on moist germination paper. Seed germination is affected by the dehydration treatment in a manner similar to that described in FIG. 1. The remaining seeds were imbibed in an Agrobacterium tumefaciens culture prepared as explained in Example 1. The seeds with approximately 15% moisture content are imbibed with an Agrobacterium solution, removed from the imbibition culture and are transferred to Petri dishes containing solid MS medium supplemented with 2% sucrose and incubated for 2 days, in the dark at RT. After this period, the seeds are transferred to either solid or liquid MS medium supplemented with 500 mg/L carbenicillin or 300 mg/L cefotaxime to kill the Agrobacterium. Once the seedlings have produced roots, they are transferred to sterile soil. The medium of the regenerated plants is washed off before transferring the plants to soil. The plants are kept under a plastic cover for 1 week to favor the acclimatization process. The plants are then transferred to a growth room. Seeds are collected and germinated and screened for herbicide resistant progeny.

Expression of the uidA gene is determined in the surviving T1 plants as in Example 1. Samples of the T1 transgenic plants are analyzed by PCR to confirm the presence of T-DNA. These results are confirmed by Southern hybridization in which DNA is electrophoresed on a 1% agarose gel and transferred to a positively charged nylon membrane (Roche Diagnostics). The PCR DIG Probe Synthesis Kit (Roche Diagnostics) is used to prepare a digoxigenin-labelled probe by PCR, and is used as recommended by the manufacturer.

Inheritance and expression of the T-DNA is confirmed in the T2 and subsequent generations by spraying the plants with a selection agent, such as a Basta or Arsenal herbicide, depending on the selectable marker used in the T-DNA. The transgene is stably transmitted to the T2 and subsequent generations in a Mendelian manner.

### Example 4

Dessication Leads to Higher Levels of Agrobacterium Transformation

The experimental protocol was the same as described in Example 1. One set of soybean embryo axes was not dried in order to reproduce the method of Graves and Goldman (1986 Plant Mol. Biol. 7:43-50) and U.S. Pat. No. 5,376,543. Another set of soybean embryo axes was dried as described in Example 1. A reporter gene, such as uidA, was used to monitor the integration of the T-DNA into the plant genome as in Example 1. The TO tissues generated with both methods were evaluated for GUS expression. While the tissues that were dessicated according to Example 1 demonstrated GUS expression, the T0 tissues generated using the Graves and Goldman method did not express the GUS reporter gene. In addition, the T1 seeds collected from the T0 primary transgenic plants are germinated and sprayed with Basta or Arsenal herbicides in accordance with the resistance gene in the binary vector used for transformation. Some of the seedlings from the dessicated embryo axes survive after spraying with the herbicide, but none of the seedlings from the hydrated embryo treatment survive. This shows that the method of Graves and Goldman is not effective and that a desiccation treatment followed by imbibition of the Agrobacterium provides for a more suqcessful transformation method.

### Example 5

A Comparison of Agrobacterium and Direct Uptake of DNA as Vectors for DNA Delivery

This example demonstrated imbibition of dehydrated soybean zygotic embryo axes in an Agrobacterium suspension culture and compared it with a solution containing naked plasmid DNA molecules. A reporter gene (uidA or GFP) was used to evaluate the integration of the DNA into the plant genome.

The experimental protocol for the preparation of dry soybean embryo axes was the same as described in Example 1. One set of soybean embryo axes was imbibed in a culture of Agrobacterium tumefaciens. Another set was imbibed in a solution of DNA as described by Senaratna et al. (1991 Plant Science, 79:223-228). A reporter gene, such as uidA, was used to monitor the integration of the T-DNA into the plant genome as in Example 1. The T1 seeds collected from the T0 primary transgenic plants are germinated and sprayed with Basta or Arsenal herbicides depending on the resistance gene in the binary vector used for transformation. Seedlings from the dessicated embryo axes treated with Agrobacterium culture survive after spraying with the herbicide, however, none of the seedlings from the naked DNA treatment survive. This shows that the method of Senaratna et al. is not effective and that desiccation treatment followed by imbibition of the Agrobacterium provides for a more successful transformation method. Similar results are obtained with Canola and Arabidopsis seeds treated as described in Examples 2 and 3.

### Example 6

Desiccated Somatic Embryos as an Alternative to Zygotic Embryos

The transformation methods described above are applicable to any plant material that can be dried and rehydrated to produce meristematic tissue. To illustrate this principle, somatic embryos are induced from an embryogenic genotype of alfalfa (Medicago sativa) as described by McKersie and Bowley (1998 Somatic Embryogenesis: Forage Improvement using Synthetic Seeds and Plant Transformation. In: Molecular and Cellular Technologies for Forage Improvement, Eds. E C Brummer, N S Hill and CA Roberts. Crop Science Society of America Special Publication number 26). Petiole explants are induced for 2 weeks on SHk solid medium (Shetty and McKersie, 1993 Plant Science 88: 185-193) containing 1 mg/L 2,4-D and 0.2 mg/L kinetin, and then transferred to liquid B5 medium and finally to BOi2Y solid medium without growth regulators. Tolerance of desiccation is induced as described in McKersie and Bowley (1998, supra) and U.S. Pat. No. 5,238,835. The dry somatic embryos are imbibed in a solution of Agrobacterium tumefaciens as described in Example 1.

Samples of the primary transgenic plants (T0) are analyzed by PCR to confirm the presence of T-DNA. Inheritance and expression of the T-DNA is confirmed in the T1 generation by testing leaves from each of the plants for activity of the uidA reporter gene. These results are confirmed by Southern hybridization as described previously. Only a small number of the T1 plants contain the T-DNA but these are easily selected by spraying the seedlings with a selection agent, such as a Basta or Arsenal herbicide, depending on the selectable marker used in the T-DNA. The transgene is stably transmitted to the T2 and subsequent generations in a Mendelian manner.

1. A method of preparing a plant zygotic embryo for transformation comprising:
a) imbibing the plant zygotic embryo in an aqueous solution; and
b) dehydrating the plant zygotic embryo.

2. The method of claim 1, wherein the plant zygotic embryo of step a) is a plant seed.

3. The method of claim 1, wherein the plant zygotic embryo of step a) and of step b) is a plant seed.

4. The method of claim 1, wherein the plant zygotic embryo is imbibed for a period of approximately 15-24 hours, and wherein the plant zygotic embryo is dehydrated to a moisture content of approximately 10-25%.

5. The method of claim 1, wherein the plant zygotic embryo is derived from a plant selected from the group consisting of a legume, and a canola plant.

6. The method of claim 1, wherein the plant zygotic embryo is derived from a soybean plant.

7. The method of claim 1, wherein the plant zygotic embryo is from a monocot.

8. The method of claim 1, wherein the plant zygotic embryo is from a dicot.

9. The method of claim 1, wherein the plant zygotic embryo is imbibed for no more than 48 hours.

10. The method of claim 1, wherein the plant zygotic embryo is imbibed for approximately 15-24 hours.

11. The method of claim 1, wherein the plant zygotic embryo is imbibed for approximately 18 hours.

12. The method of claim 1, wherein the plant zygotic embryo is dehydrated to a moisture content of less than 60%.

13. The method of claim 1, wherein the plant zygotic embryo is dehydrated to a moisture content of approximately 10-25%.

14. The method of claim 1, wherein the plant zygotic embryo is dehydrated to a moisture content of less than approximately 20%.

15. The method of claim 1, wherein the aqueous solution comprises water.

16. The method of claim 1, wherein the aqueous solution consists essentially of water.

17. A method of preparing a plant somatic embryo for transformation comprising dehydrating the plant somatic embryo.

18. The method of claim 17, wherein the plant somatic embryo is dehydrated to a moisture content of less than 60%.

19. The method of claim 17, wherein the plant somatic embryo is dehydrated to a moisture content of less than approximately 20%.

20. The method of claim 17, wherein the plant somatic embryo is derived from a plant selected from the group consisting of a soybean plant, and a canola plant.

21. The method of claim 17, wherein the plant somatic embryo is from a monocot.

22. The method of claim 17, wherein the plant somatic embryo is from a dicot.

23. A method of transforming a plant zygotic embryo, comprising the steps of:
a) imbibing the plant zygotic embryo in an aqueous solution;
b) dehydrating the plant zygotic embryo; and
c) imbibing the plant zygotic embryo in an Agrobacterium solution wherein the Agrobacterium comprises a transgene.

24. The method of claim 23, wherein the plant zygotic embryo is imbibed for a period of approximately 15-24 hours, and wherein the plant zygotic embryo is dehydrated to a moisture content of approximately 10-25%.

25. The method of claim 23, wherein the plant zygotic embryo is imbibed for approximately 18 hours.

26. The method of claim 23, wherein the plant zygotic embryo is dehydrated to a moisture content of less than approximately 20%.

27. The method of claim 23, wherein the aqueous solution comprises water.

28. The method of claim 23, wherein plant zygotic embryo is imbibed in the Agrobacterium solution for at least 30 minutes.

29. The method of claim 23, wherein the plant zygotic embryo is imbibed in the Agrobacterium solution for approximately two hours.

30. The method of claim 23, wherein the Agrobacterium is selected from the group consisting of C58C1, pMP90 and LBA4404.

31. The method of claim 23, wherein the transgene encodes a protein that alters the phenotype of the transformed plant.

32. The method of claim 23, wherein the transgene is an herbicide resistance transgene.

33. The method of claim 23, wherein after imbibing the plant zygotic embryo in the Agrobacterium solution, the plant zygotic embryo is transferred to an essentially Agrobacterium free incubation medium.

34. The method of claim 33, wherein the incubation medium is a MS medium.

35. The method of claim 34, wherein the incubation medium is selected from the group consisting of a solid and a semi-solid medium.

36. The method of claim 33, further comprising a subsequent step of treating the plant zygotic embryo with an Agrobacterium inhibiting agent.

37. The method of claim 36, wherein the Agrobacterium inhibiting agent is selected from the group consisting of timentin, carbenicillin and cefotaxime.

38. The method of claim 33 or 36, further comprising a subsequent step of transferring the plant embryo to a further growth medium.

39. The method of claim 38, wherein the further growth medium comprises a selection agent.

40. A method of transforming a plant somatic embryo, comprising
a) dehydrating a plant somatic embryo; and
b) imbibing the plant somatic embryo in an Agrobacterium solution wherein the Agrobacterium comprises a transgene.

41. The method of claim 40, wherein the plant somatic embryo is dehydrated to a moisture content of less than 60%.

42. The method of claim 40, wherein the plant somatic embryo is dehydrated to a moisture content of less than approximately 20%.

43. The method of claim 40, wherein plant somatic embryo is imbibed in the Agrobacterium solution for at least 30 minutes.

44. The method of claim 40, wherein the plant somatic embryo is imbibed in the Agrobacterium solution for approximately two hours.

45. The method of claim 40, wherein the transgene encodes a protein that alters the phenotype of the transformed plant.

46. The method of claim 40, wherein the transgene is an herbicide resistance transgene.

47. The method of claim 40, wherein after imbibing the plant somatic embryo in the Agrobacterium solution, the plant embryo is transferred to an essentially Agrobacterium free incubation medium.

48. The method of claim 47, further comprising a subsequent step of treating the plant embryo with an Agrobacterium inhibiting agent.

49. The method of claim 48, wherein the Agrobacterium inhibiting agent is selected from the group consisting of timentin, carbenicillin and cefotaxime.

50. A transgenic plant or plant tissue produced by the method of claim 23 or claim 40.
